(19) United States
(12) Patent Application Publication
(10) Pub. No.: US 2024/0350027 A1
(43) Pub. Date: Oct. 24, 2024

(54) Methods and Apparatus for Benchtop Metabolite Profiling in Situ

(71) Applicant: ViBo Health Inc., Los Angeles, CA (US)

(72) Inventor: Gil Travish, Los Angeles, CA (US)

(21) Appl. No.: 18/683,473

(22) PCT Filed: Aug. 15, 2022
(86) PCT No.: PCT/US2022/040369

§ 371 Date: Feb. 13, 2024

Related U.S. Application Data
(60) Provisional application No. 63/234,138, filed on Aug. 17, 2021.

(51) Int. Cl. A61B 5/055; A61B 5/145; G01R 33/34

(52) U.S. Cl. CPC A61B 5/055; A61B 5/14546; G01R 33/34092

(57) ABSTRACT

Described herein are systems, devices, and methods for characterization of metabolite compounds utilizing magnetic resonance spectrometers. In one embodiment, a system for characterization of metabolite compounds is provided, the system comprising a magnetic resonance spectrometer configured to generate an in vivo magnetic resonance dataset; a radio frequency transmitter and a radio frequency detector, wherein the radio frequency detector detects a signal from the tissue volume that is used to generate the in vivo magnetic resonance dataset; a processor operably coupled to the magnetic resonance spectrometer; and a memory operably coupled to the processor providing instructions to the processor to extract at least one metabolomic parameter from the in vivo magnetic resonance dataset, wherein the at least one metabolomic parameter relates to a concentration of at least one metabolite within the tissue volume.

### CROSS-REFERENCE TO RELATED APPLICATION

The current application claims priority to U.S. Provisional Patent Application No. 63/234,138 filed on Aug. 17, 2021, the disclosure of which is incorporated herein by reference.

### FIELD OF THE INVENTION

The present invention generally relates to spectroscopy and more specifically to methods and apparatus for metabolite profiling.

### BACKGROUND

Routine diagnostics represent a massive burden on healthcare and yet have traditionally been limited to a narrow set of Point of Care (POC) tests, laboratory tests (ex vivo), or imaging. Standard in-vitro lab tests can help elucidate a patient's health and diagnose several conditions; but cost, time, and patient discomfort results in these tests being performed infrequently and being limited to suspected morbidities. Metabolite profiling can involve invasive tests such as blood draws and collection of urine samples which are then typically analyzed offline. Moreover, these samples are strictly collected during a subject's visit to their healthcare provider, leading to infrequent sampling. Infrequent sampling can result in missing diagnoses due to poor timing. Thus, less invasive in-vivo metabolite profiling methods are needed, which can collect metabolite data from a subject in areas outside of a traditional healthcare setting.

References of interest may include: U.S. Pat. Nos. 6,943,033; 8,064,982; 9,316,709; US2013/049867A1; US2014/0287936A1; US2017/0007148A1; Nguyen, et al., Real-Time In-Organism NMR Metabolomics Reveals Different Roles of AMP-Activated Protein Kinase Catalytic Subunits, Analytical Chemistry 2020, 92 (11), 7382-7387; Percival, et al., Benchtop NMR Spectroscopy as a Potential Tool for Point-of-Care Diagnostics of Metabolic Conditions: Validation, Protocols and Computational Models. High-Throughput 2019, 8, 2; Markley, et al., The future of NMR-based metabolomics, Current Opinion in Biotechnology, 43, 2017, 34-40;

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 is a perspective view of an example of a metabolite profiling kiosk device (may also be referred to as “magnetic resonance spectrometer” or “bench-top magnetic resonance spectrometer”), according to embodiments described herein.

FIG. 2 illustrates the magnetic fields produced by a Halbach magnet array and examples of such a magnet array, according to embodiments herein.

FIG. 3 illustrates an overview schematic of a benchtop device for monitoring the health status of a subject, according to embodiments herein.

FIG. 4 illustrates a block diagram of a user's interaction with the user interface of a metabolomic profiling kiosk or device, according to embodiments herein.

FIG. 5 illustrates a schematic diagram of a control system for any of the devices of FIG. 1-3, according to embodiments herein.

FIG. 6 illustrates a block diagram of a scan procedure of any of the devices of FIG. 1-3, according to embodiments herein.

FIG. 7 illustrates a schematic of a division of a sample into subvolumes for targeting, according to embodiments herein.

FIG. 8A illustrates a block diagram of a targeting procedure, according to embodiments herein.

FIG. 8B illustrates segmentation of a sample, according to embodiments herein.

FIG. 9 illustrates a schematic detailing a variety of locations and methods by which a user can interact with any of the devices of FIG. 1-3, according to embodiments herein.

FIG. 10 illustrates a schematic of a repeated workflow and interaction of a user with the health monitoring system for trend monitoring, according to embodiments herein.

FIG. 11 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

Sample: As used herein, sample refers to an analyte material contained in a biological matrix, such as an extremity of a subject or patient.

Benchtop: As used herein, benchtop describes an object or device which has a volumetric size and a weight, which allow the object or device to be placed on top of a table or bench.

Low-resolution: As used here, low-resolution refers to a device having a resolving power that is insufficient to achieve baseline separation of frequency-domain resonance peaks of one or more molecules of interest without the need to perform data processing steps beyond simple Fourier transform of the free-induction-decay data.

Low-field magnet: As used herein, a low-filed magnet refers to a magnet or magnet array capable of producing a magnetic field that does not exceed 3 T.

Magnet array: as used herein, a magnet array is used to refer to an arrangement of one or more permanent or electromagnets, which are used to provide a magnetic field within a specific volume.

Focus: as used herein, focus can be used to connote a control of the location and/or size of an RF field, in addition to the plain meanings of the term.

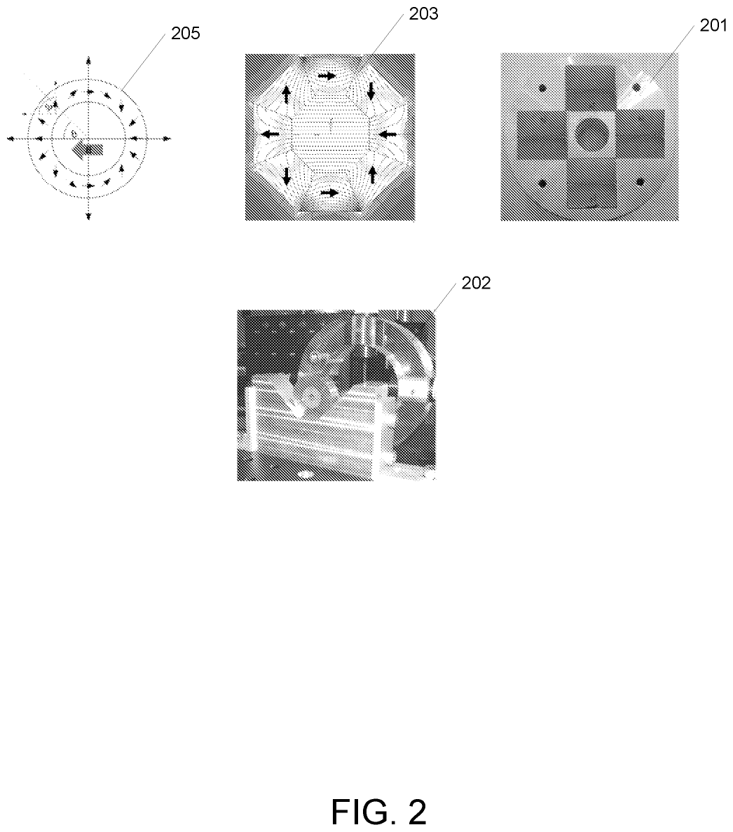

The present disclosure relates to metabolite profiling, especially through in-vivo nuclear magnetic resonance spectroscopy measurements in a benchtop device (may also be referred to as a “magnetic resonance spectrometer”). The present disclosure further relates to systems and methods for characterizing metabolite compounds. In one aspect, the system may comprise a benchtop device for monitoring the health of a subject over time. In some embodiments, the benchtop device comprises a magnet, an RF pulse generator, and an RF pickup or antenna. In some embodiments, the antenna is a phased-array antenna. In some embodiments, the phased array is configured to allow spectroscopic selection of a target volume contained within a larger volume. In some embodiments, the magnet can be a permanent magnet, a cryogen-free superconducting magnet, a pulsed magnet, or a combination of two or more thereof. In some embodiments the magnet is optionally a Halbach magnet array (illustrated in FIG. 2). In some embodiments, the device further comprises a blood oxygen monitor such as a pulse oximeter, which measures the saturation of hemoglobin and pulse rate optically, through a translucent part of an appendage of a subject, or in reflectance mode at the surface of a subject's appendage.

A benchtop device or object may be described as having a volume in the range of 1 cm³ and 6 m³ and a weight in the range of 1 g to 1000 kg. Example metabolites that may be measured are outlined in Table 1.

TABLE 1
Example metabolites or biomarkers that may be measure by some embodiments of the device described herein.
Metabolite or biomarker

| | | |
|---|---|---|
| H+ | UMP | L-Leucine |
| H2O | CTP | L-Histidine |
| ATP | S-Adenosyl-L-methionine | Hexadecenoyl-ACP (n- |
| | | C16:1ACP) |
| Phosphate | L-Cysteine | 1-Pyrroline-5-carboxylate |
| ADP | alpha-D-Ribose 5-phosphate | Sulfite |
| Nicotinamide adenine dinucleotide | UDPglucose | Carbamoyl phosphate |
| Nicotinamide adenine dinucleotide - | 5,6,7,8-Tetrahydrofolate | D-Erythrose 4-phosphate |
| reduced | | |
| CO2 | Acetoacetyl-ACP | 3-Phospho-D-glycerate |
| Nicotinamide adenine dinucleotide | UDP | CDPdiacylglycerol |
| phosphate | | |
| Pyruvate | UTP | Xanthosine 5′-phosphate |
| Nicotinamide adenine dinucleotide | Succinyl-CoA | dTTP |
| phosphate - reduced | | |
| L-Glutamate | L-Threonine | L-Tyrosine |
| Coenzyme A | Putrescine | L-Homocysteine |
| ammonium | Glycine | dATP |
| AMP | GMP | 10-Formyltetrahydrofolate |
| Acetyl-CoA | Spermidine | R-3-hydroxy-myristoyl-ACP |
| 2-Oxoglutarate | IMP | Octadecenoyl-ACP (n- |
| | | C18:1ACP) |
| acyl carrier protein | Phosphatidylglycerol | Palmitoyl-ACP (n- |
| | | C16:0ACP) |
| Phosphoenolpyruvate | L-Arginine | (S)-Dihydroorotate |
| L-Aspartate | L-Lysine | Tetradecenoyl-ACP (n- |
| | | C14:1ACP) |
| L-Glutamine | 5,10- | 2-Oxobutanoate |
| | Methylenetetrahydrofolate | |
| Glyceraldehyde 3-phosphate | Chorismate | 5-Amino-1-(5-Phospho-D- |
| | | ribosyl)imidazole-4- |
| | | carboxamide |
| CMP | D-Alanine | L-Phenylalanine |
| Glycerol 3-phosphate | L-Proline | phosphatidylserine |
| GTP | L-Asparagine | UDP-2,3-bis(3- |
| | | hydroxytetradecanoyl)glu- |
| | | cosamine |
| 5-Phospho-alpha-D-ribose 1-diphosphate | UDP-N-acetyl-D- | ferritin |
| | glucosamine | |
| Dihydroxyacetone phosphate | D-Glucosamine 6-phosphate | L-Homoserine |
| L-Alanine | Nicotinate D-ribonucleotide | Undecaprenyl phosphate |
| L-Serine | dGTP | (R)-Pantothenate |
| D-Fructose 6-phosphate | Iminoaspartate | Orotate |
| Malonyl-[acyl-carrier protein] | D-Ribulose 5-phosphate | L-Aspartate 4-semialdehyde |
| D-Glucose 1-phosphate | Myristoyl-ACP (n- | Sedoheptulose 7-phosphate |
| | C14:0ACP) | |
| GDP | 3-Methyl-2-oxobutanoate | N2-Formyl-N1-(5-phospho- |
| | | D-ribosyl)glycinamide |
| Oxaloacetate | L-Methionine | 5-Methyltetrahydrofolate |
| Reduced thioredoxin | L-Tryptophan | Hydrogen sulfide |
| FAD | dTMP | 7,8-Dihydrofolate |
| D-Glucose 6-phosphate | phosphatidate | dTDP |
| Oxidized thioredoxin | L-Valine | glycogen |
| 5-amino-1-(5-phospho-D- | D-Glycero-D-manno- | 3-Deoxy-D-manno-2- |
| ribosyl)imidazole | heptose 1-phosphate | octulosonate |
| KDO(2)-lipid IV(A) | D-erythro-1-(Imidazol-4- | ADP-L-glycero-D-manno- |
| | yl)glycerol 3-phosphate | heptose |
| N1-(5-Phospho-D-ribosyl)glycinamide | Sulfate | beta-Alanine |
| 5-[(5-phospho-1-deoxyribulos-1- | UDP-3-O-(3- | D-Alany1-D-alanine |
| ylamino)methylideneamino]-1-(5- | hydroxytetradecanoyl)-N- | |
| phosphoribosyl)imidazole-4-carboxamide | acetylglucosamine | |
| Phosphatidylglycerophospha te | 2-Amino-4-hydroxy-6-(D- | O-Succinyl-L-homoserine |
| | erythro-1,2,3-trihydroxypropyl)- | |
| | 7,8-dihydropteridine | |
| UDP-N-acetylmuramoyl-L-alanine | UDP-3-O-(3- | Quinolinate |
| | hydroxytetradecanoyl)-D- | |
| | glucosamine | |
| N-Succinyl-LL-2,6-diaminoheptanedioate | Orotidine 5′-phosphate | 2-(Formamido)-N1-(5-phospho- |
| | | D-ribosyl)acetamidine |
| 2-Dehydro-3-deoxy-D-arabino-heptonate 7- | UDP-N-acetyl-3-O-(1- | D-Arabinose 5-phosphate |
| phosphate | carboxyvinyl)-D-glucosamine | |
| O-Phospho-L-serine | 6-hydroxymethyl dihydropterin | 1-(5-Phosphoribosyl)-5-[(5- |
| | | phosphoribosylamino)methylidene- |
| | | amino]imidazole-4- |
| | | carboxamide |
| UDP-N-acetylmuramate | O-Acetyl-L-serine | lipopolysaccharide |
| (S)-2-[5-Amino-1-(5-phospho-D- | UDP-N-acetylmuramoyl-L- | 6-hydroxymethyl- |
| ribosyl)imidazole-4-carboxamido]succinate | alanyl-D-glutamyl-meso-2,6- | dihydropterin pyrophosphate |
| | diaminopimeloyl-D-alanyl-D- | |
| | alanine | |
| UDP-N-acetylmuramoyl-L- | UDP-N-acetylmuramoyl-L- | Shikimate |
| alanyl-D-gamma-glutamyl- | alanyl-D-glutamate | |
| meso-2,6-diaminopimelate | | |
| 3-Carboxy-3-hydroxy-4-methylpentanoate | Cardiolipin | Undecaprenyl-diphospho-N- |
| | | acetylmuramoyl-L-alanyl-D- |
| | | glutamyl-meso-2,6- |
| | | diaminopimeloyl-D-alanyl-D- |
| | | alanine |
| 2,3-Bis(3- | (R)-2,3-Dihydroxy-3- | Undecaprenyl-diphospho-N- |
| hydroxytetradecanoyl)-beta-D- | methylbutanoate | acetylmuramoyl-(N- |
| glucosaminyl 1-phosphate | | acetylglucosamine)-L-ala-D-glu- |
| | | meso-2,6-diaminopimeloyl-D-ala- |
| | | D-ala |
| 3-Deoxy-D-manno-octulosonate 8- | (S)-2-Acetolactate | 5-phosphoribosyl-5- |
| phosphate | | carboxyaminoimidazole |
| N-((R)-4- | 3′-Phosphoadenylyl sulfate | 3-Carboxy-2-hydroxy-4- |
| Phosphopantothenoyl)-L-cysteine | | methylpentanoate |
| Peptidoglycan subunit of Escherichia coli | 1-(5-Phosphoribosyl)-ATP | N-Acetyl-D-glucosamine 1- |
| | | phosphate |
| Adenosine 5′-phosphosulfate | 2,3,2′3′-Tetrakis(beta- | L-Cystathionine |
| | hydroxymyristoyl)-D- | |
| | glucosaminyl-1,6-beta-D- | |
| | glucosamine 1,4′-bisphosphate | |
| Dihydropteroate | Shikimate 5-phosphate | (S)-3-Methyl-2-oxopentanoate |
| 3-Dehydroshikimate | (R)-Pantoate | 5-O-(1-Carboxyvinyl)-3- |
| | | phosphoshikimate |
| 1-(5-Phosphoribosyl)-AMP | 2-Dehydropantoate | 2-Amino-4-hydroxy-6-(erythro- |
| | | 1,2,3- |
| | | trihydroxypropyl)dihydropteridine |
| | | triphosphate |
| 4-Methyl-2-oxopentanoate | CDPethanolamine | D-Glycero-D-manno-heptose |
| | | 1,7-bisphosphate |
| D-Glycero-D-manno- | 5-amino-1-(5-phospho-D- | 3-(Imidazol-4-y1)-2-oxopropyl |
| heptose 7-phosphate | ribosyl)imidazole-4-carboxylate | phosphate |
| 2,3,4,5- | 5-Phospho-beta-D-ribosylamine | Pantetheine 4′-phosphate |
| Tetrahydrodipicolinate | | |
| 3-Phosphohydroxypyruvate | N-Succinyl-2-L-amino-6- | N-Carbamoyl-L-aspartate |
| | oxoheptanedioate | |
| 3,4-dihydroxy-2-butanone 4-phosphate | D-4′-Phosphopantothenate | (R)-2,3-Dihydroxy-3- |
| | | methylpentanoate |
| KDO(2)-lipid IV(A) with laurate | Diphosphate | Malonyl-CoA |
| 2-Isopropylmaleate | Succinate | 4-amino-4-deoxychorismate |
| KDO-lipid IV(A) | Ubiquinol-8 | D-Glutamate |
| ADP-D-glycero-D-manno-heptose | Menaquinone 8 | 3-(4- |
| | | Hydroxyphenyl)pyruvate |
| KDO(2)-lipid (A) | Ubiquinone-8 | L-Histidinol |
| N6-(1,2-Dicarboxyethyl)-AMP | Fumarate | Deamino-NAD+ |
| 5-Formamido-1-(5-phospho-D- | L-Malate | (S)-2-Aceto-2-hydroxybutanoate |
| ribosyl)imidazole-4-carboxamide | | |
| Lipid A Disaccharide | Glyoxylate | N-(5-Phospho-D- |
| | | ribosyl)anthranilate |
| 2,3-Dihydrodipicolinate | FADH2 | N(omega)-(L-Arginino)succinate |
| 3-Dehydroquinate | Ornithine | 2,5-Diamino-6-(ribosylamino)-4- |
| | | (3H)-pyrimidinone 5′-phosphate |
| 4-Phospho-L-aspartate | Citrate | 5-Amino-6-(5′- |
| | | phosphoribosylamino)ura cil |
| S-Adenosylmethioninamine | Isocitrate | Anthranilate |
| 3-Carboxy-4-methyl-2-oxopentanoate | N-Acetyl-L-glutamate | Riboflavin |
| | 5semialdehyde | |
| Phenylpyruvate | C′-(3-Indolyl)-glycerol 3- | FMN |
| | phosphate | |
| D-Glucosamine 1-phosphate | 6,7-Dimethyl-8-(1-D- | L-Citrulline |
| | ribityl)lumazine | |
| Dephospho-CoA | N-Acetyl-L-glutamyl 5- | 3-Phospho-D-glyceroyl phosphate |
| | phosphate | |
| ADPglucose | N2-Acetyl-L-ornithine | 5-Amino-6-(5′- |
| | | phosphoribitylamino)ura cil |
| 4-Aminobenzoate | D-Glycerate 2-phosphate | 1-(2-Carboxyphenylamino)-1- |
| | | deoxy-D-ribulose 5-phosphate |
| L-Histidinol phosphate | N-Acetyl-L-glutamate | Dihydroneopterin monophosphate |
| LL-2,6- | glucose | Na+ |
| Diaminoheptanedioate | | |
| K+ | chloride | Hemoglobin |
| insulin | TSH | Uric acid |
| Lactic acid | urea | Creatinine |
| calcium | albumin | bilirubin |
| Alkaline phosphatase | Aspartate aminotransferase | Alanine aminotransferase |
| HDL or LDL cholesterol or triglycerides | thyroxine | Vitamin B12 |
| Methylmalonic acid | | |

Measured metabolites may include biomarkers relevant to longevity, frailty, and health-span which may include one or more of: Vitamin B, Vitamin E, Vitamin C, carnitines, triglycerides, GlycA, cholesterol, Lysine, isocitrate, or combinations thereof including derivatives and related metabolites. Measured metabolites may include biomarkers associated with trauma status and may include one or more of: Hemoglobin (Hb), creatinine, hematocrit, blood sugar, urea (BUN), glial fibrillary acidic protein (GFAP), ubiquitin C-terminal hyrdrolase-L1 (UCH-L1), S100B protein or combinations thereof. Measured metabolites may include biomarkers of acidosis or related conditions which may include measurement of nucleoside triphosphates (NTP) or phosphocreatine (PCr) or combinations thereof. Measured metabolites may include small molecules or proteins and may further include treatment or abuse compounds such as morphine.

Measured metabolites may include biomarkers indicative of fitness or lack thereof, for example, by measurement of changes in fat metabolism in response to exercise. Metabolites including free fatty acids, acylcarnitines and glycerol may increase after exercise. Measurement of Glutamine (GLN) and/or glutamate (GLU) may provide a method for monitoring fatigued states in subjects. For example, decreased levels of glutamine and glutamine/glutamate ratios may be associated with fatigue and suboptimal training capacity. Measurement and subsequent adjustment of Omega-3 index (OM3I) in subjects may be useful to augment both health and athletic performance. For example, increasing OM3I from ˜4.5 to ˜6% in subjects through supplementation may enhance cycling efficiency. Measured metabolites may include indicators of generalized health of a subject such as isoleucine, al-acid glycoprotein, VLDL cholesterol, triglycerides, glucose, fatty acid profiles, GlycA, high-density lipoproteins, or combinations of one or more thereof.

In some embodiments, the device is operably coupled to at least one processer configured to automatically select the target volume within a larger volume based on a series of spectra acquisitions. The device can comprise an NMR detector machine capable of targeting specific areas within a large volume. The large volume may be a bore sized and shaped to receive a tissue volume of a user, where the tissue volume may include an appendage or appendages, such as fingers, toes, hands, feet, arms, or legs. The NMR machine may be a low-resolution NMR detector, wherein a low-field magnet is employed. One aspect of the present disclosure includes the combination of the NMR device together with a database containing metabolomics data. An automated scan of a subject's metabolomic profile may be taken. The automated scan can include a first set of scans, which are used in autonomous targeting to identify regions of interest (ROI) s, where targeted subvolumes are measured. These scans may be performed rapidly to minimize the time required for a complete automated scan. Examples of targeted subvolumes may include areas which are composed primarily of a target tissue type. Target tissue types may include bone, fat, blood vessels, or a combination of two or more thereof. In many embodiments, a subvolume may include a portion of a predetermined volume. In general, any volume may be divided into a plurality of subvolumes. In some embodiments, subvolumes may not be equal in size or shape to each other. In some embodiments, a sum of all the subvolumes may equal the volume under consideration.

In some embodiments, a device is used to measure a series of NMR spectra over an automatically determined volume of interest. The specific spectra obtained can be a function of a series of sampling spectra taken throughout the region of interest, where these sampling spectra are used to determine the most likely regions for a predetermined set of tissue or fluid types. A second set of spectra can be measured within each of those tissue type regions. These measurement spectra can be used to fit data about metabolite concentrations.

In some embodiments, the low field magnet may have a field strength of about 0.1 T to about 3 T. In some embodiments, the low field magnet may have a field strength of about 0.1 T to about 0.5 T, about 0.1 T to about 1 T, about 0.1 T to about 1.5 T, about 0.1 T to about 2 T, about 0.1 T to about 2.5 T, about 0.1 T to about 3 T, about 0.5 T to about 1 T, about 0.5 T to about 1.5 T, about 0.5 T to about 2 T, about 0.5 T to about 2.5 T, about 0.5 T to about 3 T, about 1 T to about 1.5 T, about 1 T to about 2 T, about 1 T to about 2.5 T, about 1 T to about 3 T, about 1.5 T to about 2 T, about 1.5 T to about 2.5 T, about 1.5 T to about 3 T, about 2 T to about 2.5 T, about 2 T to about 3 T, or about 2.5 T to about 3 T. In some embodiments, the low field magnet may have a field strength of about 0.1 T, about 0.5 T, about 1 T, about 1.5 T, about 2 T, about 2.5 T, or about 3 T. In some embodiments, the low field magnet may have a field strength of at least about 0.1 T, about 0.5 T, about 1 T, about 1.5 T, about 2 T, or about 2.5 T. In some embodiments, the low field magnet may have a field strength of at most about 0.5 T, about 1 T, about 1.5 T, about 2 T, about 2.5 T, or about 3 T.

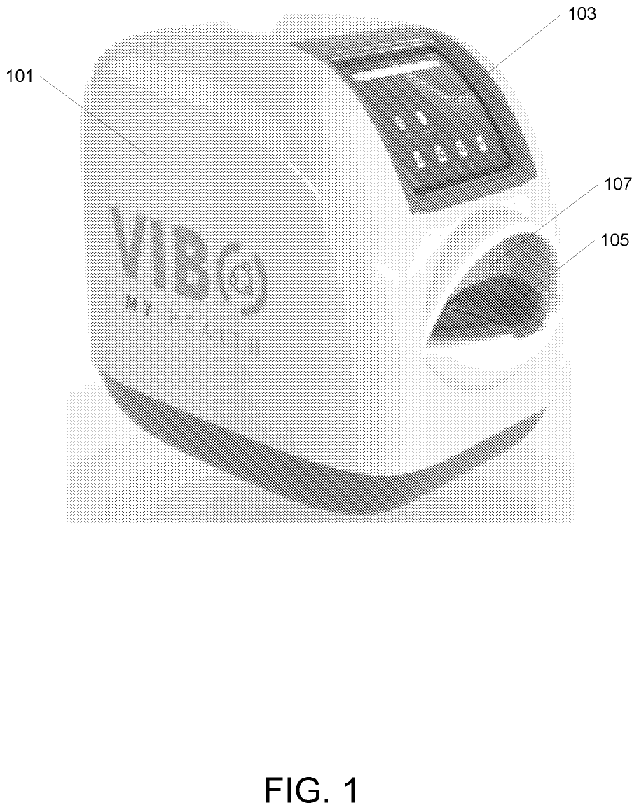

An example metabolic profiling kiosk device 101 is detailed in FIG. 1. The device 101 may comprise an NMR spectrometer and a user interface device 103 through which a user or subject may interact with the metabolite profiling kiosk device 101. Upon interaction with the user interface 103, the subject may be prompted to insert an appendage, such as their hand, into the magnet bore of the spectrometer through an entrance passage 105 and a safety gate 107 of the kiosk device 101.

An example of a magnet, which may be employed in a device such as, but not limited to, the device described in FIG. 1 is detailed in FIG. 2. In the example of FIG. 2, the magnetic field used by the device 101 is provided by a permanent magnet Halbach array such as those seen in 201, 202. Magnetic field simulations 203 and magnetic dipole calculations 205 for such an array are further detailed in FIG. 2.

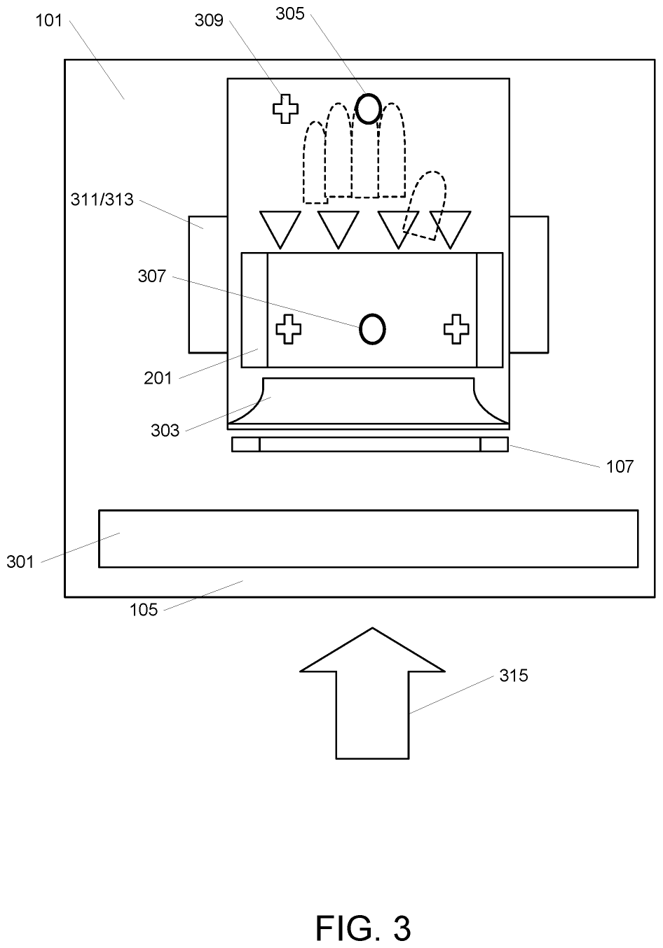

A schematic of the metabolic profiling kiosk device 101 is illustrated in FIG. 3. The safety gate 107 may be used to exclude magnetic material from entering the bore of magnet(s) 201, 203, which can provide the magnetic field needed for NMR measurements. An inspection halo 301 can allow visual inspection of safety gate 107 and may further comprise a metal detector and entrance to the magnet bore. When the safety gate 107 is open, a user may place 315 an appendage in the magnet bore through an entrance passage 105 and the entrance funnel 303. The user may then rest the appendage on a blood oxygen sensor 305 and a pulse rate monitor 307. One or more position sensors 309 may be used by the device 101 to determine the location of the appendage within the magnet bore. The device 101 may further determine the location of a region of interest, at least partially using data from the position sensors. At least one RF transmit antenna 311 and at least one RF receive antenna 313 may be used in the device to perform NMR measurements. The RF transmit antenna and RF receive antennas 311/313 may be a single antenna or antenna array used to perform both functions.

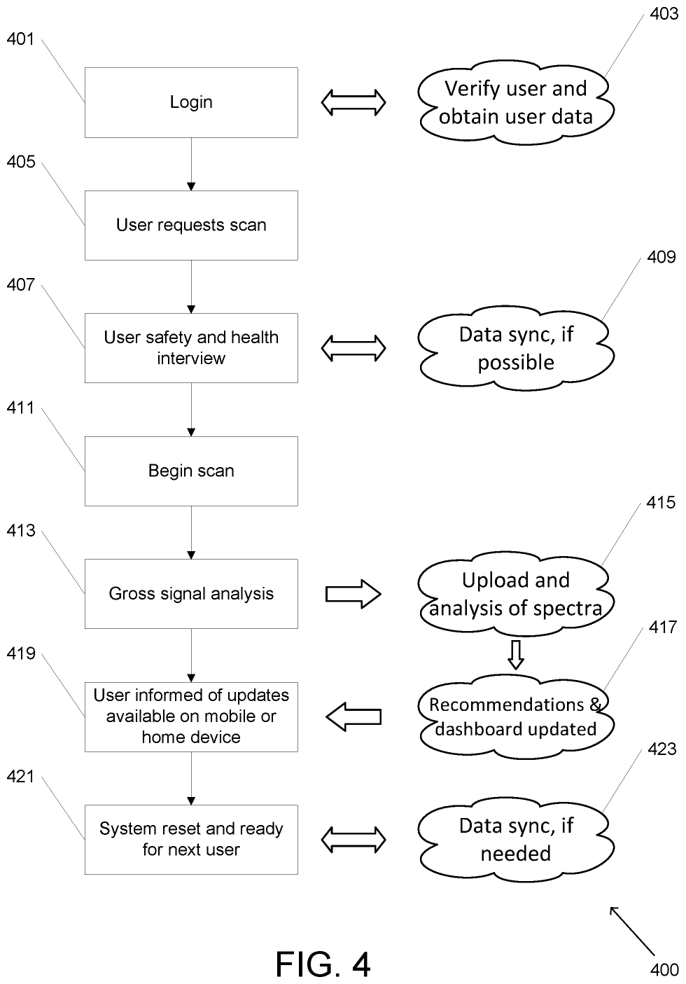

A schematic diagram of a user or subject's interaction with the example kiosk device 101 in a method 400 is outlined in FIG. 4. Upon initial interaction, the user may be prompted by the user interface 103 to login or create an account in a step 401. Login credentials may be verified by comparison with credentials stored in a database, and associated user data may be obtained from the database by the kiosk in a step 403. The user may then request a scan in a step 405, which may trigger the kiosk to prompt the user to answer safety and health interview questions in a step 407. Data from screening questions may be synced with or stored in the database for later use in a step 409, and the user may be prompted to begin the scan in a step 411. The user may insert an appendage through the entrance funnel 107, to the bore of the magnet. A scan may begin causing data to be collected by the kiosk, which may feed the data into a gross signal analysis routine in a step 413. Analysis may be performed either locally by the kiosk or remotely for example on a server. Data may be uploaded to a database. Acquired data may be compared to data retrieved or stored in a database in a step 415, and recommendations and reports of trends may be displayed on a dashboard accessible by the user in a step 417. Updated recommendations and reports may also be delivered to the user through an interface on a mobile or home device in a step 419. At the conclusion of an interaction, the kiosk device may be reset and may be made ready to interact with the next user in a step 421, and data or control software may be synchronized with a database in a step 423.

Although the above steps show method 400 of interacting with a kiosk device 101 in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as advantageous to the method 400.

One or more steps of the method 400 may be performed with circuitry as described herein, for example, one or more of the processor of the kiosk 101 or any computing device in communication with the kiosk 101. The circuitry may be programmed to provide one or more steps of the method 400, and the program may comprise program instructions stored on a computer readable memory or programmed steps of the circuitry.

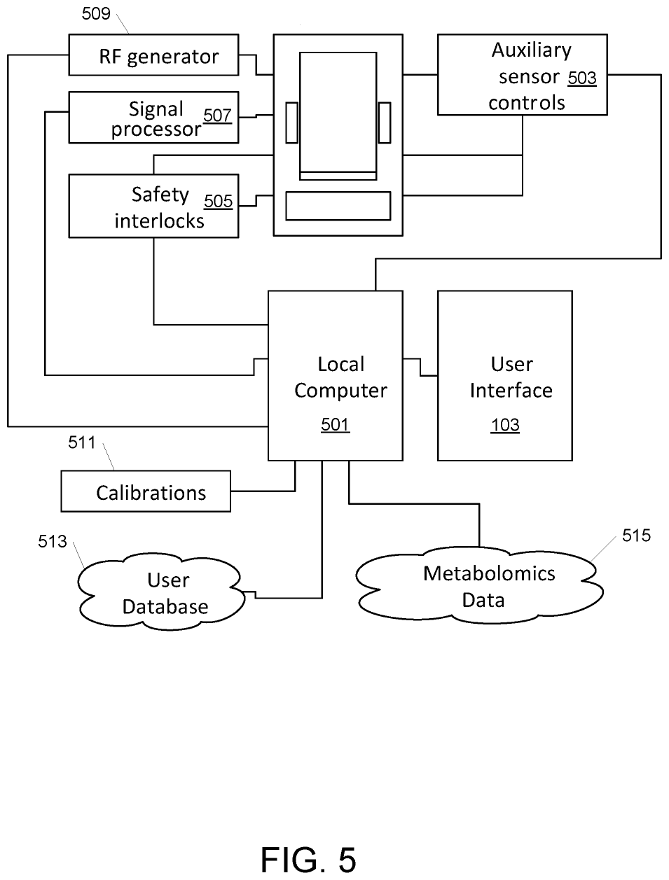

A schematic diagram detailing the interaction of kiosk hardware with a local computer and one or more databases is detailed in FIG. 5. The example kiosk device 101 may comprise subsystems which may be controlled by or interact with a local computer system 501 such as a workstation, a personal computer, a laptop computer, a tablet computer, a smart phone, or the like in communication with the kiosk device 101. These subsystems may include a user interface 101, one or more auxiliary sensors such as blood oxygen monitors, position sensors, and/or metal detectors 503, one or more safety interlocks 505, one or more signal processors 507 operably coupled to one or more RF antennas, one or more RF generators 509 operably coupled to one or more RF antennas, and/or one or more hardware or software calibrations 511. The local computer system 501 may further interact with one or more local or remote databases, which may store user data 513, metabolomics data 515, calibration data 511, or a combination of one or more thereof.

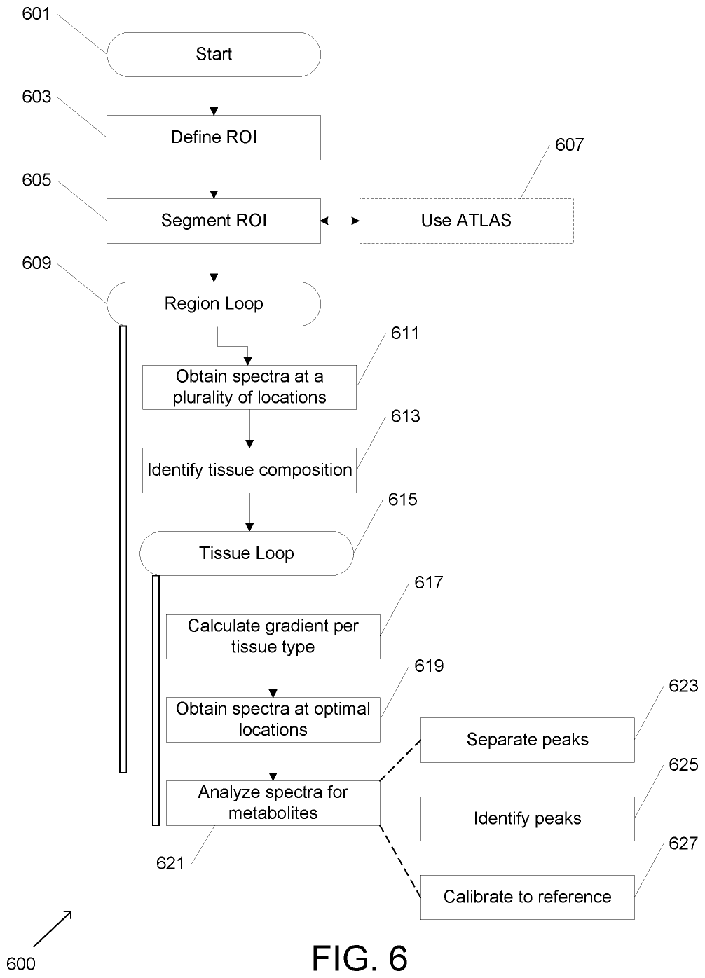

A block diagram of a procedure 600 that may be employed by the example device 101 for performing a targeted NMR scan and analyzing acquired data is shown in FIG. 6. The scan routine may be started in a step 601 either through the user interface 103 or automatically upon detection of a trigger event such as an appendage being placed in the bore of the magnet. A subset of data received from the auxiliary sensor systems 503 may be used to define an initial region of interest (ROI) in a step 603, which may be used to select initial scan points. The ROI may be further segmented into smaller regions in a step 605 optionally using a database of calibration data (an atlas) 607 to locate regions with a high likelihood of containing the analytes of interest. The segmented ROIs may be processed by a region loop in a step 609, which may comprise one or more the steps of: obtaining NMR spectra at two or more locations within a subvolume in a step 611; identifying tissue composition corresponding to the obtained spectra by comparison with calibration data optionally retrieved from a database in a step 613; and processing each identified tissue type using a tissue loop in a step 615, which may further comprise one or more of the steps of: calculating a gradient per tissue type in a step 617 to identify one or more regions of high relative concentration of a tissue of interest; obtaining NMR spectra at optimal locations (the regions of high relative concentration) in a step 619; and analyzing obtained spectra for metabolites in a step 621, which may further comprise one or more of the steps of: processing data to resolve peaks in a step 623; identifying the peaks in a step 625; and calibrating the spectra to are reference in a step 627.

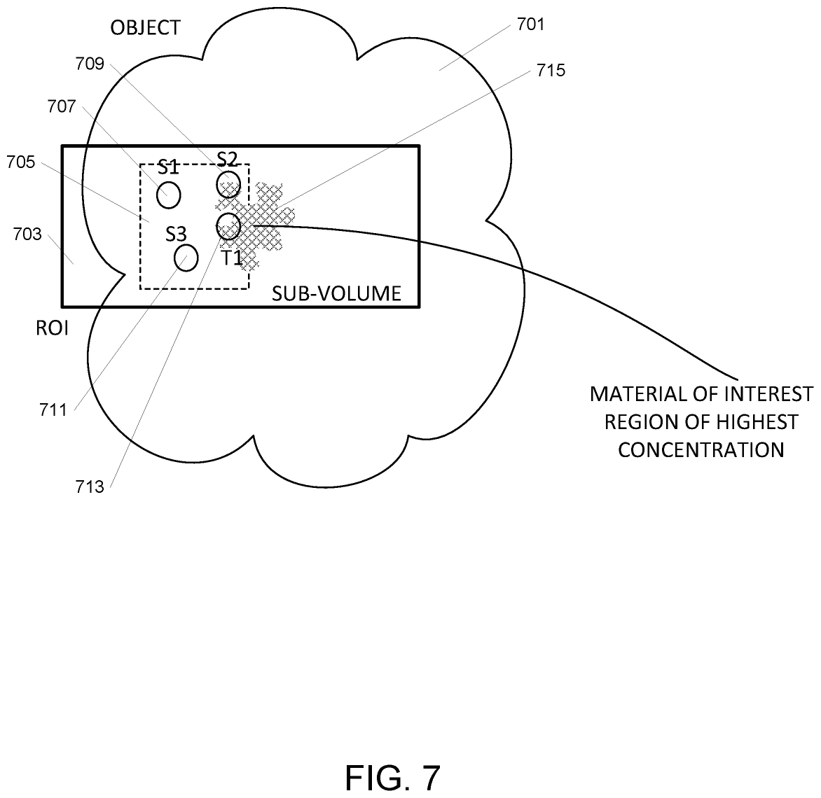

A schematic further detailing the above described targeting procedure 600 is seen in FIG. 7. A user may place an appendage into bore of the magnet of an example kiosk. The NMR spectrometer of the example kiosk may allow scan volumes to be selected over a range of space 701. Sensor data may be used to determine an initial position of a region of interest (ROI) 703 which is divided into smaller subvolumes 705. Spectra may initially be taken at two or more initial locations, but preferably spectra may be acquired in at least three locations 707, 709, 711. Data collected at initial locations may be analyzed to determine a tissue gradient, which may further be used to locate a subvolume 713 likely to be located in an area of high relative concentration 715 of the tissue or analyte of interest.

Although the above steps show targeting procedure 600 in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as advantageous to the method 600.

One or more steps of the method 600 may be performed with circuitry as described herein, for example, one or more of the processor of the kiosk 101 or any computing device in communication with the kiosk 101. The circuitry may be programmed to provide one or more steps of the method 600, and the program may comprise program instructions stored on a computer readable memory or programmed steps of the circuitry.

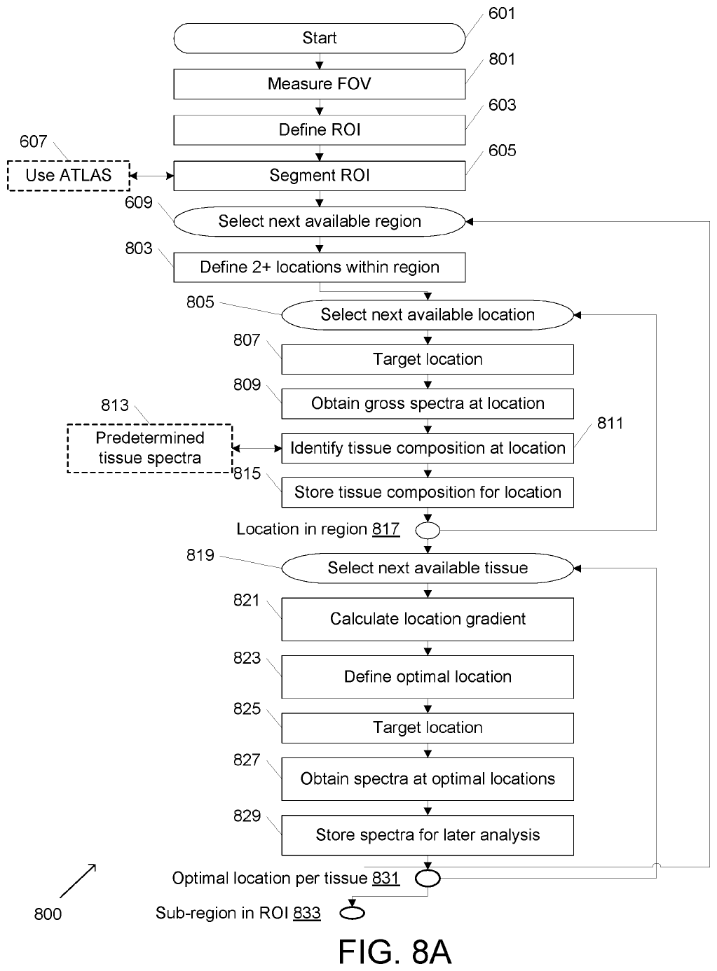

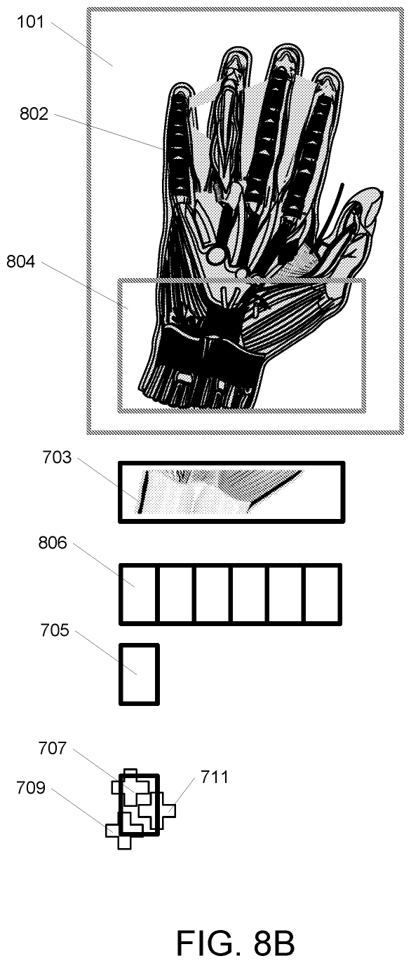

An expanded block diagram of a procedure 800 that may be employed by the example device for performing a targeted NMR scan and analyzing acquired data is shown in FIG. 8A. The scan routine may be started in the step 601 either through the user interface 103 or automatically upon detection of a trigger event such as an appendage being placed in the bore of the magnet. A subset of data received from the auxiliary sensor systems 503 may be used to define an initial region of interest (ROI) in the step 603, which may be used to select initial scan points. FIG. 8B illustrates an example segmentation procedure demonstrating segmentation of an appendage containing a plurality of tissue types 802. In some examples field of view (FOV) data 804 from an integrated camera may be used to determine the initial ROI in a step 801. The ROI may be further segmented into smaller regions (segments 806) in a step 605 optionally using a database of calibration data (an atlas) 607 to locate regions with a high likelihood of containing the analytes of interest. The segmented ROIs may be processed by a region loop in a step 609, which may comprise one or more the steps of: defining two or more locations within the segmented ROI in a step 803; iteratively selecting a location from the defined locations in a step 805; for each selected location: targeting the location in a step 807; obtaining gross spectra at the location in a step 809; comparing the obtained spectra with a database or calibration data in a step 813 to identify the tissue composition at the location in a step 811; and storing information about the tissue type at that location in a step 815. This process may result in information concerning the types of tissues present in a plurality of subvolume locations in a step 817, which may then be used to identify locations of a target tissue type. A list of predetermined target tissue types may be used to acquire measurements of metabolites specific to those tissue types. For each predetermined tissue type, the scan procedure may comprise one or more of the following steps: select a tissue type in a step 819; calculate a location gradient in a step 821 from data acquired at a plurality of subvolume locations; determine from the location gradient an optimum subvolume point for the predetermined tissue type in a step 823, where an optimum point may be a point predicted to contain the highest relative concentration of a selected target tissue within a subvolume; target the optimum point in a step 825; obtain spectra at the optimum point in a step 827; and store the spectra for later analysis in a step 829; The result of this procedure may be the generation of a plurality of optimal points for each tissue type and corresponding NMR spectra within a given subvolume in a step 831, and the entire process or portions thereof may be repeated for each segmented volume of the initial ROI to generate a list of the optimums within a plurality of subregions for a plurality of tissue types in a step 833.

Although the above steps show method 800 for performing a targeted NMR scan and analyzing acquired data in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as advantageous to the method 800.

One or more steps of the method 800 may be performed with circuitry as described herein, for example, one or more of the processor of the kiosk 101 or any computing device in communication with the kiosk 101. The circuitry may be programmed to provide one or more steps of the method 800, and the program may comprise program instructions stored on a computer readable memory or programmed steps of the circuitry.

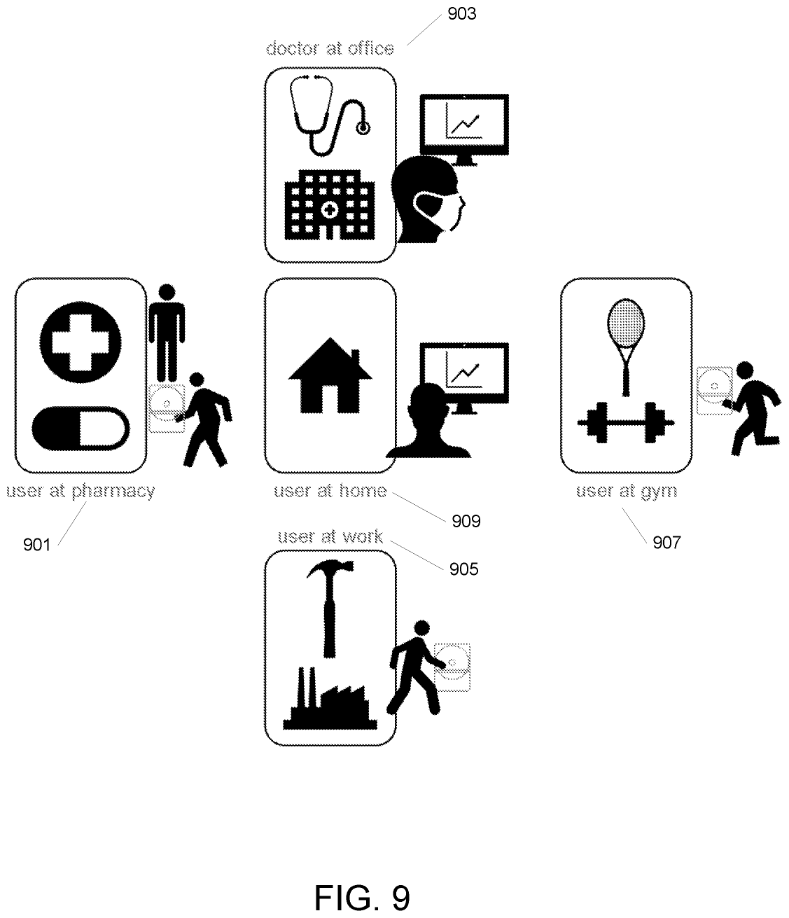

A user or subject and/or their healthcare providers may interact with an example embodiment of a metabolic profiling kiosk system in a number of settings, as seen in FIG. 9. For example, the same individual may visit a pharmacy 901 and perform a scan at an example kiosk device. The scan results may be sent to the user's designated healthcare provider, which may be useful for health advising or diagnosis in a doctor's office 903. The user may later perform a subsequent scan using a different kiosk located in their place of employment 905, or in a gym 907. The user may then interact with a dashboard on a home or mobile device, which may allow them to track trends and may further provide health recommendations 909.

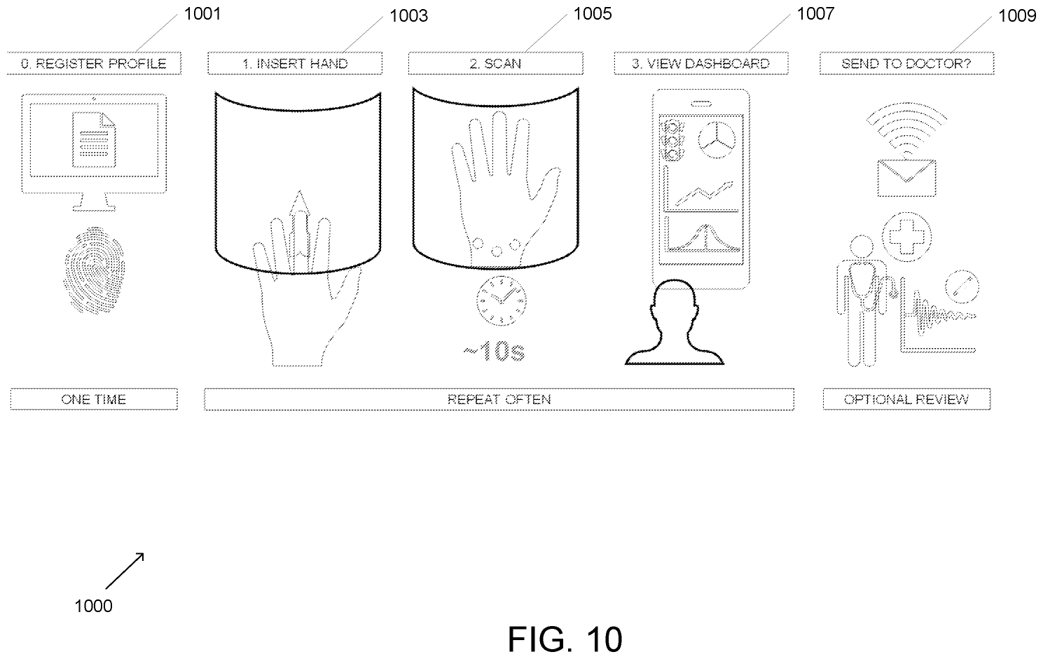

A preferred example sequence 1000 of interacting with the example kiosk system is outlined in FIG. 10. Once per session, a user's identity may be confirmed or they may be registered as a new user, and a diagnostic selection may be made (for example they may choose to perform a new scan) in a step 1001. If a scan is selected, the scan may be further configured in a step 1003. Preferably, a user repeats the scans frequently in a step 1005, so they may be presented with a dashboard which may track their health status over time in a step 1007. The user may then have the option to have the data reviewed by a doctor or health professional, either in person or via tele-health in a step 1009.

Although the above steps show method 1000 for tracking a subject's metabolomic profile in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as advantageous to the method 1000.

One or more steps of the method 1000 may be performed with circuitry as described herein, for example, one or more of the processor of the kiosk 101 or any computing device in communication with the kiosk 101. The circuitry may be programmed to provide one or more steps of the method 1000, and the program may comprise program instructions stored on a computer readable memory or programmed steps of the circuitry.

### Example 1: Measurement of Metabolites and Small-Molecule Biomarkers in a Subject

In a preferred embodiment, the device is enclosed in a free-standing kiosk with a touch-screen, keypad, or other input device interface for interacting with a user, see FIG. 1. The user approaches the kiosk, similar to the one detailed in FIG. 3 and is invited to sign into an existing, or sign up for a new, health tracking account. A block diagram of the user's interaction with the user interface is illustrated in FIG. 4 with method 400. If the user has an existing account, presenting a phone, RFID device, NFC device, or similar NRF device to a reader on the kiosk allows the user to sign in without further interaction with the user interface. The user is asked a series of questions to establish safety to use the device and if any significant health events have occurred since the last time the user was scanned or within the recent past. The user is reminded to remove all jewelry, watches, and other magnetic items. The control algorithms may be tightly coupled to the hardware since the software must respond to the specific sample (i.e. the spectra being measured) in order to perform targeting steps, as well as safety checks, and reading of sensor data. An overview schematic of the relationship of instrument hardware to the computer control is shown in FIG. 5. Once the user confirms they have done so, they are instructed to place their hand into an aperture once a safety gate opens.

The user introduces their hand into an opening, a metal detector checks for the presence of ferrous materials and the user is allowed to place their hand into the aperture if no ferrous material is detected. The user rests their hand on a support within the bore of an NMR magnet. The user interface prompts the user to remain still for several seconds. A schematic diagram of the major operations of the scan procedure is illustrated in FIG. 6 with method 600. A camera or infrared sensor identifies the location of the user's wrist and determines an initial region of interest (ROI) or volume of interest to scan. The ROI is divided into a plurality of subregions, as illustrated in FIG. 7. A sequence of initial NMR scans is taken within each subregion. These scans may vary in the period and in the number of times they are repeated in order to obtain a signal to noise level above a predetermined threshold for each set of scans in regions matching the spectral profile of a predetermined tissue type.

Pulse, hand movement, and blood oxygen are simultaneously monitored, and if anomalies suggesting excess movement are detected the user is asked to repeat the test. Each scan involves applying power to a set of RF antennas, sweeping the RF frequency across a transmit probe, and detecting and processing signals from the receive probe, where both probes consist of phased array antennas. To achieve improved spectral targeting, gradient coils can be used in combination with phased array transmit and receive coils. This combination allows localizing both input RF power (transmit) and the sensing (receive) to improve the signal-to-noise ratio. Gradient coils used do not require a perfectly homogenous magnetic field over the volume.

Targeting methods are used to improve signal to noise ratios. A schematic overview of the data acquisition process is illustrated in FIG. 8A with method 800. Target tissue types may include fluids and can further include skin, fat (adipose), muscle, bone, blood or sweat, or interstitial fluid. A search volume is determined (sometimes called a region of interest (ROI)) through mechanical means, optical means, or a combination thereof. The search volume is segmented into smaller volumes of interest (VOI) s. Two or more points in each volume of interest are targeted within each region and obtain spectra for each VOI. Tissue gradients are calculated for a set of predetermined tissue types (e.g. skin, fat, bone, blood) and their associated spectra to identify the tissue types present in the VOIs. The location with the likelihood of highest concentration of a targeted tissue is determined by triangulation of the points within a VOI. For each target tissue type and triangulated location, a spectrum is obtained and recorded. Optionally, the strongest tissue type in each region can be selected and the process is repeated for each VOI region in order to speed up the data acquisition. In the event that multiple points for the same tissue type are acquired and/or some points have a mix of tissue types, scans are ranked to determine the best points (highest percentage of a target tissue type and best signal-to-noise) which are used for further analysis.

The resulting signal at the receive probe is recorded as a raw spectrum. After all scans of all predetermined tissue types are complete or after a maximum predetermined length of time has elapsed, the scans end and the user is informed to remove their hand. Spectra are pre-processed locally, including hardware calibration correction and other processing steps to improve the signal-to-noise ratio. A data set (consisting of all collected spectra and metadata) is compiled, compressed and uploaded to a database, which is optionally a cloud-based database. The kiosk automatically signs off the user and closes the safety gates and resets the user interface to a welcome screen.

The uploaded data is processed to determine relative concentrations of a predetermined set of analytes, which can include metabolites, biomarkers and related small molecules. The processing may involve machine learning or multiparametric fitting in order to deconvolve overlapping spectra. If this is not the user's first interaction with the system, data from previous scans of the same user may be used to aid in the calibration. The processed data is saved in the database and a subset of the data is made available to the user in a simplified dashboard, which may be accessed directly from the terminal, through a web-interface, or through an application on a mobile device (see FIG. 9). The processed data containing the user's current metabolic profile is compared to any previous profiles of the same user stored in the database to evaluate individual trends. The user's data is further compared to population averaged profiles aggregated from a plurality of user's data stored in the database to determine their health status relative to the general population.

If significant changes or trends are detected which indicate health or disease states, the user is notified of these trends. If the trends or changes are beyond a predetermined alarm threshold, the user is advised to contact a clinician. If the trends or changes are beyond a critical alarm threshold, the user is presented with warning menu advising their health status and that emergency services will be contacted if they fail to refuse such contact within a predetermined time period. If the user fails to respond to the prompt, emergency services are called, and an audible alarm is triggered. If they choose to dismiss the prompt, they are sent a reminder to follow up with a clinician.

Independent of the identified trends, the user is prompted to upload data to a clinician of their choice. The user continues to visit various kiosks periodically and data trends are established (FIG. 10). During each subsequent visit, and through the web-interface, mobile application, etc. detected positive or negative changes and trends are reported to the user.

### Computer Systems

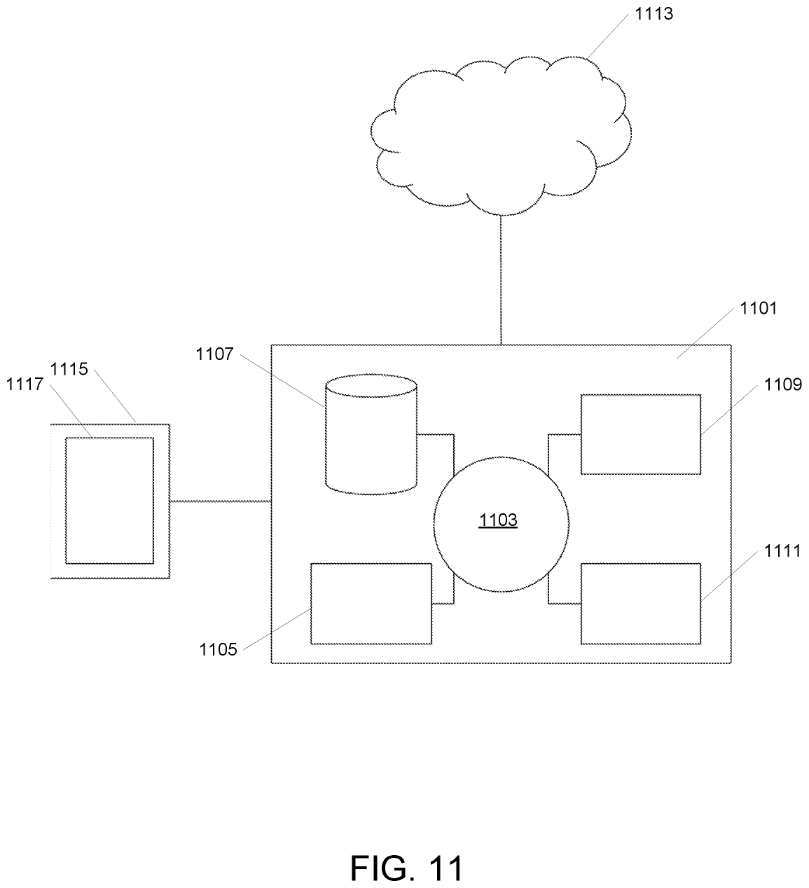

The present disclosure provides computer systems that are programmed to implement methods of the disclosure. FIG. 11 shows a computer system 1101 that is programmed or otherwise configured to perform the above described functions of a benchtop metabolic profiling device, or the above described functions of interacting with a user to provide health data originating from a benchtop metabolic profiling device. The computer system 1101 can regulate various aspects of the subsystems of the present disclosure, such as, for example, controlling or reading data from sensors, controlling or reading back data from RF generators or RF antennas, controlling or performing spectroscopic targeting. The computer system 1101 can be an electronic device of a user or a computer system that is remotely located with respect to the electronic device. The electronic device can be a mobile electronic device. The electronic device may be a wearable electronic device such as a smartwatch.

The computer system 1101 includes a central processing unit (CPU, also “processor” and “computer processor” herein) 1103, which can be a single core or multi core processor, or a plurality of processors for parallel processing. The computer system 1101 also includes memory or memory location 1105 (e.g., random-access memory, read-only memory, flash memory), electronic storage unit 1107 (e.g., hard disk), communication interface 1109 (e.g., network adapter) for communicating with one or more other systems, and peripheral devices 1111, such as cache, other memory, data storage and/or electronic display adapters. The memory 1105, storage unit 1107, interface 1109 and peripheral devices 1111 are in communication with the CPU 1103 through a communication bus (solid lines), such as a motherboard. The storage unit 1107 can be a data storage unit (or data repository) for storing data. The computer system 1101 can be operatively coupled to a computer network (“network”) 1113 with the aid of the communication interface 1109. The network 1113 can be the Internet, an internet and/or extranet, or an intranet and/or extranet that is in communication with the Internet. The network 1113 in some cases is a telecommunication and/or data network. The network 1113 can include one or more computer servers, which can enable distributed computing, such as cloud computing. The network 1113, in some cases with the aid of the computer system 1101, can implement a peer-to-peer network, which may enable devices coupled to the computer system 1101 to behave as a client or a server.

The CPU 1103 can execute a sequence of machine-readable instructions, which can be embodied in a program or software. The instructions may be stored in a memory location, such as the memory 1105. The instructions can be directed to the CPU 1103, which can subsequently program or otherwise configure the CPU 1103 to implement methods of the present disclosure. Examples of operations performed by the CPU 1103 can include fetch, decode, execute, and writeback.

The CPU 1103 can be part of a circuit, such as an integrated circuit. One or more other components of the system 1101 can be included in the circuit. In some cases, the circuit is an application specific integrated circuit (ASIC).

The storage unit 1107 can store files, such as drivers, libraries and saved programs. The storage unit 1107 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1113. For instance, the computer system 1101 can communicate with a remote computer system of a user (e.g., through a mobile app, web interface, or other means of access using a personal computer system). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1101 via the network 1113.

Methods as described herein can be implemented by way of machine (e.g., computer processor) executable code stored on an electronic storage location of the computer system 1101, such as, for example, on the memory 1105 or electronic storage unit 1107. The machine executable or machine readable code can be provided in the form of software. During use, the code can be executed by the processor 1103. In some cases, the code can be retrieved from the storage unit 1107 and stored on the memory 1105 for ready access by the processor 1103. In some situations, the electronic storage unit 1107 can be precluded, and machine-executable instructions are stored on memory 1105.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1115 that comprises a user interface (UI) 1117 for providing, for example, a means of interacting with a metabolic profiling device in any of the manners described herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1103. The algorithm can, for example, include scan targeting procedures including but not limited to triangulation between measured points to find an optimum point.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A system for characterization of metabolite compounds, the system comprising:
a magnetic resonance spectrometer comprising:
a bore, wherein the bore is sized and shaped to receive a tissue volume of a user, wherein the magnetic resonance spectrometer generates an in vivo magnetic resonance dataset; and
a radio frequency (RF) transmitter and a RF detector, wherein the RF detector detects a signal from the tissue volume in response to a transmitted RF signal from the RF transmitter, and wherein the signal from the tissue volume is used in generating the in vivo magnetic resonance dataset;
a processor operably coupled to the magnetic resonance spectrometer; and
a memory operably coupled to the processor providing instructions to the processor to extract at least one metabolomic parameter from the in vivo magnetic resonance dataset, wherein the at least one metabolomic parameter relates to a concentration of at least one metabolite within the tissue volume of the user.

2. The system of claim 1, wherein the magnetic resonance spectrometer comprises a volume less than one meter cubed and a weight less than 25 kilograms.

3. The system of claim 1, further comprising a magnetic array, wherein the magnetic array produces a dipole magnetic field.

4. The system of claim 3, wherein the magnetic array comprises an electromagnet or a permanent magnet.

5. The system of claim 3, wherein the magnetic array comprises a Halbach arrangement.

6. The system of claim 1, wherein the radiofrequency transmitter creates a radiofrequency waveform, wherein the radiofrequency waveform comprises a variable spectral and temporal profile set at least partly based on a set of parameters selected by a user.

7. The system of claim 6, further comprising at least one antenna wherein the at least one antenna is configured to at least partially control a focus of the radiofrequency waveform and wherein the at least one antenna is a phased array antenna further comprising a plurality of radiofrequency transmitters.

8. The system of claim 1, further comprising a radiofrequency detector, wherein the radio frequency detector detects a signal from the tissue volume in response to transmitted radiofrequency signal from the radiofrequency transmitter.

9. The system of claim 1, further comprising a user interface, wherein the user interface provides the user with instructions to use the magnetic resonance spectrometer substantially without instruction from a healthcare provider.

10. The system of claim 1, further comprising a kiosk comprising a user interface.

11. The system of claim 1, further comprising a display comprising a user interface.

12. The system of claim 1, wherein the magnetic resonance spectrometer is configured to direct data from the user to a cloud computing system.

13. The system of claim 12, wherein the data comprises the at least one metabolomic parameter paired with at least one measurement time or user identity information.

14. The system of claim 1, wherein the at least one metabolomic parameter comprises at least one metabolite concentration level, wherein the at least one metabolite concentration level comprises blood concentration levels for at least one of acetic acid, acetate, acetoacetate, adenine triphosphate, alanine, apolipoprotein, carnitine, cholesterol, choline, citrate, creatine, fatty acids, glucose, glutamate, glutamic acid, glutamine, glutathione, glutathione disulfide, glycerol, glycine, glycoprotein acetyls, histidine, 3-hydroxyl butyrate, isoleucine, lactate, lactic acid, leucine, proline, pyruvate, taurine, tryptophan, tyrosine, urea, or valine.

15. The system of claim 14, wherein the cholesterol comprises at least one of: VLDL, LDL, HDL, HDL2, HDL3, or free esterified remnant cholesterol.

16. The system of claim 14, wherein the apolipoprotein comprises at least one of: apolipoprotein A1 or apolipoprotein B.

17. The system of claim 1, wherein the magnetic resonance spectrometer is a low-field magnetic resonance spectrometer configured to generate a magnetic field less than 3 Tesla.

18. A system for characterization of metabolite compounds, the system comprising:
a magnetic resonance spectrometer comprising a bore, wherein the bore is sized and shaped to receive a tissue volume of a user, wherein the magnetic resonance spectrometer generates an in vivo magnetic resonance dataset;
a processor operably coupled to the spectrometer;
a memory operably coupled to the processor providing instructions to the processor to extract one or more metabolomic parameters from the in vivo magnetic resonance dataset, wherein the one or more metabolomic parameters relate to a concentration of one or more metabolites within the tissue volume of the user; and
wherein the memory further provides instructions to the processor to implement a machine learning process or a Bayesian optimization to extract the one or metabolomic parameters from a plurality of magnetic resonance spectra generated by the spectrometer.

19. The system of claim 18, wherein the plurality of magnetic resonance spectra are insufficient to provide the one or more metabolomic parameters without use of the machine learning algorithm, or the Bayesian optimization.

20. The system of claim 18, wherein the plurality of magnetic resonance spectra comprise a resolution which is insufficient to resolve H-NMR splitting of aromatic C—H bonds, a resolution greater than 30 ppm for phosphorus NMR, a resolution greater than 10 ppm for carbon NMR, and a resolution greater than 0.5 ppm for hydrogen NMR.

21. (canceled)

22. (canceled)

23. (canceled)

24. (canceled)

25. (canceled)

26. (canceled)

27. (canceled)
